(19) United States
(12) Patent Application Publication
(10) Pub. No.: US 2003/0224474 A1
(43) Pub. Date: Dec. 4, 2003

(54) Rapid-acting drug analysis system

(76) Inventor: Mark A. Litman, Edina, MN (US)

Correspondence Address: Mark A. Litman & Associates, P.A., York Business Center, Suite 205, 3209 West 76th St., Edina, MN, 55435, US

(21) Appl. No.: 10/446,313

(22) Filed: May 28, 2003

Related U.S. Application Data
(60) Provisional application No. 60/383,840, filed on May 30, 2002.

(51) Int. Cl. C12Q 1/28

(52) U.S. Cl. 435/28

(57) ABSTRACT

A tube or pipette-type element, such as a capillary tube or other tube element carries a drug identification system for a number of hypnotic-inducing drugs or anesthetic drugs. The drug identification system provides a visual indication of at least two or more drugs present in a liquid sample. The liquid sample is drawn into one end of the tube, moves along the tube, and functional materials and/or structure in the interior of the tube provide a visual indication of the presence of targeted contaminants (e.g., drugs) in the fluid sample. The design also minimizes or prevents any reflux of materials in the tube element back into the liquid or beverage from which the sample has been taken. An additional benefit of the system is safe disposal and moderate unit cost to the public.

### BACKGROUND OF THE INVENTION

1. Field of the Invention

The analysis device for beverages described herein relates generally to a covert apparatus that imbibes or uptakes a liquid sample for analysis. More particularly, the invention relates to a beverage analysis device that extracts a liquid sample from a beverage by capillary forces, surface tension in pores, or absorption, and then subjects the sample to a responsive system such as an analytical system that performs a visible presumptive color assay.

2. Description of the Related Art

In the technical field of chemistry and particularly analytical chemistry there are varied means for analyzing fluids of varied types to determine particular chemical content. Among the more common systems are liquid samplers, multi-compartment test kits (e.g., such as those used for testing pH and chlorine content in swimming pools), dye indicators kits and reagent strips. Currently analytical liquid samplers and test strips are used in area's that include the fields of medicine, drug detection, water quality, industrial pollutants and generally in the environmental arena.

Diverse types of liquid samplers and reagent test strips are known to the art. The outgrowth of such devices for the rapid identification of varied chemical substances at different levels of saturation in many types of liquid and biological samples is apparent. Among the most common embodiments are for assays of water, urine, saliva, solvents, blood, and other fluid samples. The majority of these devices are handled by individuals with at least some skill level and understanding relating to their specific field of endeavor.

The analysis is documented primarily by visual means. However, lab facilities and portable instrumental methods can be employed for mechanical or automated analysis. Efficient, accurate, portable and economical liquid samplers and test strips have an advantage over the more complicated and expensive instruments.

There has become an urgent need to test all types of beverages for different substances. There has recently an increased incidence in the use of strong drugs to overpower individuals to enable the drug supplier to commit criminal cats. Although this practice goes back to the use of “Mickies” in the days of Shanghaiing sailors, the recent phenomenon of “Date Rape” compounds placed in the beverages of unsuspecting individuals has raised the bar for the complexity and the need to protect an individual against ingesting these hypnotic and dangerous substances. In addition to the worries of unprotected sex, compounds such as GHB (known generically as gamma hydroxybutyric acid), ketamine and rohypnol, when mixed with alcohol or other drugs, may lead to respiratory depression, aspiration, and even death. These compounds produce a sedative effect, amnesia, muscle relaxation, and a slowing of psychomotor responses. The pharmaceutical ROHYPNOL is widely known to the masses as “roofies”, “ruffles”, “roche”, “R-2”, “rib”, and “rope” and reportedly is quickly becoming the “date rape drug of choice.”

Rohypnol is a pharmaceutical known by the generic name flunitrazepam (a benzodiazepine). Evidently, it is a very potent tranquilizer, and similar in nature to valium (diazepam) but apparently many times stronger. Sedation from some of these drugs may occur in less than 1 minute minutes after administration and lasts for several hours (e.g., up to 12 hours with a 2 milligram dose). The drug is often distributed on the street in its original “bubble packaging” which adds an air of safety to the drug at time of purchase, even by the individual who intends to commit a criminal act. When taken time and time again, it can lead to physical and psychic dependence, which is thought to increase with both dose and duration of use.

Police departments in several parts of the United States of America, have reported that, after the ingestion of Rohypnol several young women have reported waking up in unfamiliar surroundings with unfamiliar people, and/or having actually been sexually assaulted while under the influence of the drug. Recent seizures and anecdotal reporting indicate that distribution and abuse of flunitrazepam are increasing among young people domestically, even though the drug is neither legally sold nor legally manufactured in the United States.

Sodium Gamma Hydroxybutyric (GHB), a patented pharmaceutical, is also causing alarm. The clinical uses of GHB range from drug addictions to eating disorders and numerous other afflictions. However, it is also being abused in similar fashions to that of Rohypnol. The substance GHB is a endogenous compound that induces absence-like seizures and provides sedative effects similar to that of flunitrazepam.

Reportedly GHB has sustained usage, and with the advent of a more functional and readily available tablet, a resurgence of GHB will probably occur. The substance GHB has only recently been placed under the control of the United States Food and Drug Administration and, like other hypnotic drugs, is presently available for purchase over the Internet.

These drugs when placed in an unsuspecting individual's beverages and then consumed, leave the victim highly venerable to crimes such as robbery, rape, death, and assault. The perpetrators of these crimes are from all walks of life, making this problem all the more disconcerting. Since the drugs are readily available, a strategy and device must exist for deterrence, or at least defense. The device of the present invention provides a precautionary routine to the users of the device, which substantially reduces the likelihood of becoming a victim of such crimes.

It has become evident, that in this day and age, consumption of beverages of various types presents a significant danger and, without prior testing Oust prior to consumption), there is a definite risk to ones health and safety.

The related art is crowded with test devices for a wide variety of liquid substances, the following of which are examples of the most relevant developments. U.S. Pat. No. 3,965,750 shows a liquid sampler comprised of an elongated tube having a fluid-holding well cap.

The liquid sampler shown in U.S. Pat. No. 3,994,170 provides a device with a collecting container, supplied with a slot shaped aperture in the outer wall. The device includes a spiral rotating inner wall assembly that opens or closes said aperture.

U.S. Pat. No. 4,157,664 shows a device with two telescopically disposed receptacles, forming a closed container. U.S. Pat. No. 4,625,574 shows a method and apparatus are disclosed for obtaining a field sample. Furthermore, a method and means for sealing said sample from deterioration, additional contamination, or loss of volatile components are provided. U.S. Pat. No. 5,583,044 shows a fluid sampler and testing unit. This device performs a presumptive brilliant chromophoric test. U.S. Pat. No. 5,728,076 shows an ampoule holder and actuator. The device includes a cylindrical ampoule and a handle with a bore for receiving a piston.

An apparatus with provisions for a test strip is disclosed in U.S. Pat. No. 5,611,995. The device detects a specifically reacting substance in a liquid, and is intended for home use or by nonprofessional organizations, whereby the test is usually carried out by laymen or non-skilled people.

Also known to the art, and as exemplified in U.S. Pat. No. 3,915,639 is a drug of abuse dipstick, useful in visually identifying an ion exchange in physiological fluids. A dipstick is provided in combination with a stain- producing reagent, impregnated on a substrate within a matrix of numerous ion exchange resins that are outwardly exposed to the fluid to be tested. An immersible test strip including a wick member enclosed in a fluid impervious sheath, and an aperture exposing a portion of the wick member, is illustrated by U.S. Pat. No. 4,092,115.

Also known in the art, as exemplified by U.S. Pat. No. 3,715,192 is an indicator strip useful in analytical chemical procedures. The main advantage of the construction of this device is that the capillary material which absorbs the liquid medium allows the sample to be examined quickly, thereby preventing chromatographic separation of the reagent during the absorption of the liquid media.

Despite the vast number of devices and systems for sampling and/or analyzing various fluids which have been proposed in the related art, the urgent need for the present invention has not been fulfilled. U.S. Pat. No. 6,153,147 describes a beverage analysis sample or system comprising a device that includes a housing having a main body including an analysis chamber with at least one chemical reagent composition contained therein and a vessel portion for receiving and capturing a fluid specimen taken from a beverage. The device is structured to transfer the captured fluid specimen from the vessel portion to the analysis chamber wherein the chemical reagent composition, when exposed to the fluid specimen, performs a visible presumptive color assay to determine whether undesirable substances, such as fulnitrazepam (rohypnol), sodium gamma hydroxybutyric acid, caffeine or other identified drugs, are contained in the beverage. One or more windows are provided on the housing for viewing the results of the visible presumptive color assay. The fluid specimen and one or more chemical reagent compositions remain contained within the analysis chamber to prevent contamination of the beverage as well as tampering with the captured fluid specimen. This system actually requires significant technical expertise in certain formats and does not provide indications in a sufficiently short time for the environment in which they would be used. Additionally, the device clearly implies utility for essentially simple beverages (e.g., water, single liquour content beverages, and simple (two or three component) mixed drinks. To be most effective, the testing should be accomplished in less than a minute, preferably less than 45 seconds, with significant reliability and minimum technical skills.

### SUMMARY OF THE INVENTION

A tube or pipette-type element, such as a capillary tube or other tube element carries a drug identification system for a number of hypnotic-inducing drugs or anesthetic drugs. The drug identification system provides a visual indication of at least two or more drugs present in a liquid sample. The liquid sample is drawn into one end of the tube, moves along the tube, and functional materials and/or structure in the interior of the tube provide a visual indication of the presence of targeted contaminants (e.g., drugs) in the fluid sample. The design also minimizes or prevents any reflux of materials in the tube element back into the liquid or beverage from which the sample has been taken. An additional benefit of the system is safe disposal and moderate unit cost to the public.

### BRIEF DESCRIPTION OF THE FIGURES

FIG. 1 shows a tube sampling and detection system according to one aspect of the invention.

FIG. 2 shows a tube sampling and detection system according to a second aspect of the invention.

FIG. 3 shows an alternative, non-tube indicating element using the same general phenomena as the tube construction for a rapid access testing system.

FIG. 4 shows a necklace having stems of testing elements as a decoration thereon.

### DETAILED DESCRIPTION OF THE INVENTION

A system and method are provided for analyzing beverages that can be adulterated with known and specific compounds such as, but not limited to, the pharmaceuticals: a) Sodium Gamma Hydroxybutyric Acid, known generically as GHB; and b) Flunitrazepam, known generically as Rohypnol, and c) ketamine. Furthermore, the invention can be used in one embodiment to analyze beverages where any targeted drug has been previously tested. The device of the present invention, in accordance with the several embodiments thereof, is useful for personal safety and guardianship when there is a concern about the presence of dangerous and/or unhealthy substances in one's beverage, and does not require any governmental approval as no materials are ingested and it is not a medical diagnostic process.

The present invention is directed to a sampler device for random analysis of beverages just prior to consumption to determine whether certain undesirable substances are contained in the beverage. The system may be provided in sterile packaging, or at least packaging that limits dirt, moisture, dust and physical contamination. The devise does not have to be significantly prepared for immersion within the beverage and retrieval of a fluid specimen. All that may need to be done is removal of the tube element from a container and it should be ready for use. The assembly serves as a vessel to uptake the fluid specimen; exposing at least one aperture, preferably merely an open end of the tube. In use, the device is immersed in the beverage and uptake of a fluid sample occurs. After removal from the beverage, not even closure and sealing of the device is required, although this may be done in certain constructions. The fluid specimen then migrates, by capillary forces, into a visualization area that provides a marker that the drug(s) is present or absent.

There are two related subspecies for performance of the present invention to provide a beverage analysis sampling device that performs qualitative and semi-quantitative detection. One subspecies is a purely physical chromatographic analysis that requires no chemical analysis. The other subspecies comprises presumptive color assays for particular drugs. The preference for the first subspecies is that testing may be performed with a single functional system for a multiplicity of drugs, while separate chemical functionalities must be provided for the color assay testing of multiple drugs. At the present time, each system should be capable of testing for Sodium Gamma Hydroxybutyric Acid or gamma hydroxy butyrate (GHB), Rohypnol and ketamine that may be present in a beverage.

An aspect of the invention is to provide a simple, nearly fool-proof, rapid-access indication system for the presence of contaminants in beverage samples, particularly for the presence of surreptitiously provided drugs in personal beverages. The testing provides no further contamination of the beverage.

In the drawings and in detailed description of the invention that follows, there are essentially shown and described only preferred embodiments of this invention, setting forth what is considered to be the best mode contemplated of carrying out this invention at this time. As will be realized, this invention is capable of other and different embodiments, and it's several details are capable of modification in various respects, all without departing from the spirit and scope of the invention. Accordingly, the drawings and the detailed description are to be regarded as illustrative in nature, and not as restrictive.

The present invention is directed to a beverage analysis sampler for detecting the presence, and in sum instances the level, of certain substances which may be contained in a beverage. Particularly beneficial applications of the invention include analysis of varied alcoholic and non-alcoholic beverages under suspicion of Sodium Gamma Hydroxybutric Acid GHB, ketamine or rohypnol adulteration. Additionally, the invention is useful to perform qualitative analysis of a variety of drugs in a single test in various beverages including soft drinks, coffee, tea, simple alcoholic beverages and complex alcoholic concoctions.

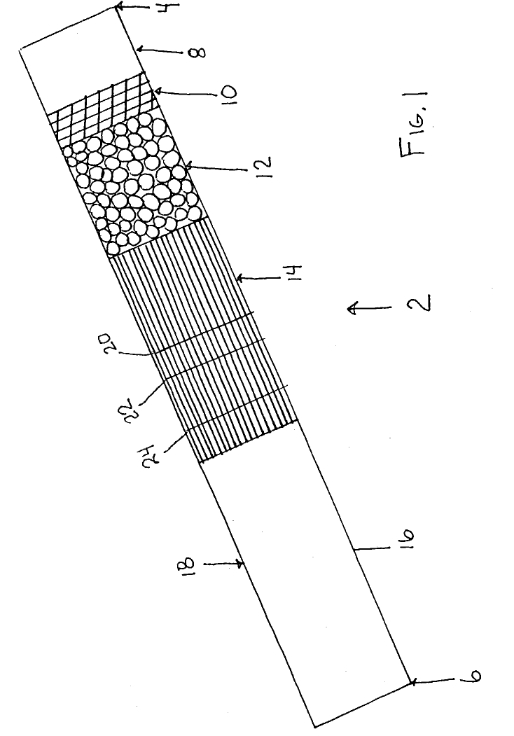

FIG. 1 shows one structural embodiment for the practice of the invention. A tube test element 2 comprises a liquid accepting end 4, and a gripping end 6. The element 2 is shown with an optional capillary zone 8 that is free of material for some distance (e.g., 0.5 to 50 mm, although greater distances may be used) to allow for the liquid to wick or be drawn into the element 2. This zone would allow removal of sample before contact by the sample with any active ingredient that might reflux back into a beverage or partial liquid sample. This zone is optional, but preferred. A gross filter 10 is also shown. This gross filter (as opposed to a molecular filter) is intended to assist in the analysis of more complex beverage concoctions by removing pulp, particulates, and other solid/undissolved materials from the liquid sample. The presence of those materials would complicate any testing procedure, without the filter being intended to chemically isolate individual chemicals for refined chemical analysis. Another component in the tube element 2 is an ingredient supply area 12, which may also be optional depending upon the present of other materials in the element 2. The ingredients may be active (reacting with drugs/chemicals in an imbibed sample) or may be passive, merely colorizing liquid flow to provide markers in a chromatographic environment. The ingredient supply area 12 may contain solid particles of the ingredient (e.g., solid iodine crystals, alcohol soluble pigments/dyes, water-soluble pigments/dyes, reactants that form dyes with specific chemicals/drugs, etc.). capsules of ingredients (e.g., microcapsules that are porous, soluble or dispersible in liquid and contain the desired ingredients in the core). Surface tension or wicking or reduced pressure applied at the gripping end 6 allows liquid to continue to pass from the ingredient supply area 12 to a display area 14. The display area 14 may, in some circumstances be combined with the ingredient supply area 12, but other constructions require a separation. One of those other constructions is the provision of a chromatographic or wicking viewing area 14. In a preferred embodiment, three marking lines 20, 22 and 24 are provided. These marking lines indicate the positions on a chromatographic path or scale where targeted drugs would appear at least after a defined time interval after immersion of the liquid accepting end 4 into the liquid to be tested. In this instance, the ingredient supply area 12 may be iodine, dye, pigments that dissolve, etc. to move along with the liquid. As patches appear on the display chromatographic area 14, chemicals in the liquid have separated for visual identification, and the dyes, pigments or iodine mark the patches for easy view. If a significant patch appears at one of the targeted marks, a drinker should at least exercise caution in the consumption of the beverage, if not call the police or notify the bar that the drink has been compromised. More sophisticated chemical analysis testing may be performed to provide scientific or forensic evidence of the presence of a specific drug. The end 16 of the tube may contain nothing or some other enhancing function, such as materials that may be wicked into the display area 14 to enhance marking or providing a second marking function that may or may not act as quickly as the rapid test (e.g., chemical reactants specific to the targeted drugs that may form specific color dyes, precipitating agents, oxidizing agents, reducing agents, fixing agents, and the like). It is important to note that the test may take as little as thirty seconds in a chromatographic display, with the chromatographic material being chosen for flow and separation characteristics. The shell 18 of the tube 2 may be rigid, flexible, or in some particular cases, compressible. For example, where ingredients in the ingredient supply area 12 are provided in frangible capsules to implement time release of ingredients, the tube may be squeezed, and the ingredient released at the appropriate time. For example, the element 2 may be dipped into the liquid, liquid enters the liquid accepting end 4, passes through the open area 8, is filtered in the filter zone 10, passes over the ingredient supply area 12, and begins to enter the display area 14. The release of ingredients from the ingredient supply area 12 may be effected at this point with frangible microcapsules by squeezing,the tube 2 around the ingredient supply area 12, rupturing capsules and releasing ingredient at the appropriate time. This is a minimally complex procedure as compared top prior art methods wherein isolation of ingredients or external application of ingredients has been required.

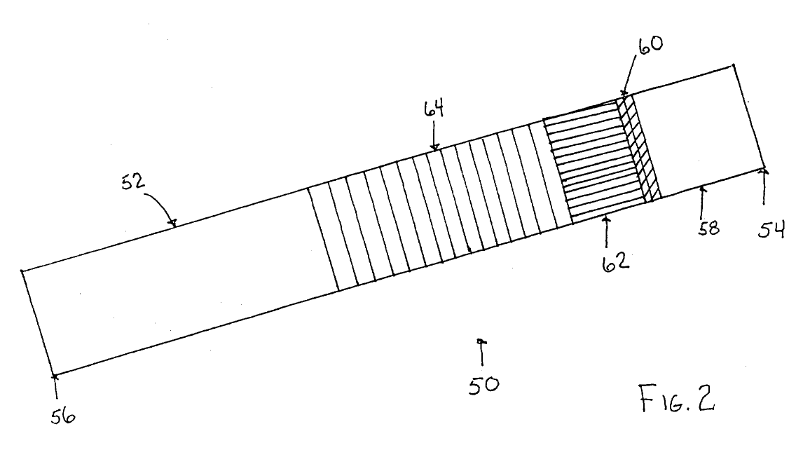

FIG. 2 shows another drug sampling system 50 comprising a shell 52 having a liquid entry end 54, and a grasping end 56. There is a open (unfilled) area 58 leading to a coarse physical filter 60, followed by a wicking or fluid advancing zone 62 (optional). A series of marked zones 64 of thin layer chromatographic material is arranged, with each zone additionally containing a chemical reagent specific for a particular target drug. It is possible to attach a rubber bulb or other element that can create a reduced pressure at the grasping end 56 of the element 2 to facilitate liquid movement. The combination of TLC marking and reagent reaction (usually to form a dye or pigment, or to cause a color change at a specific site) adds heightened assurance of the accuracy of the test.

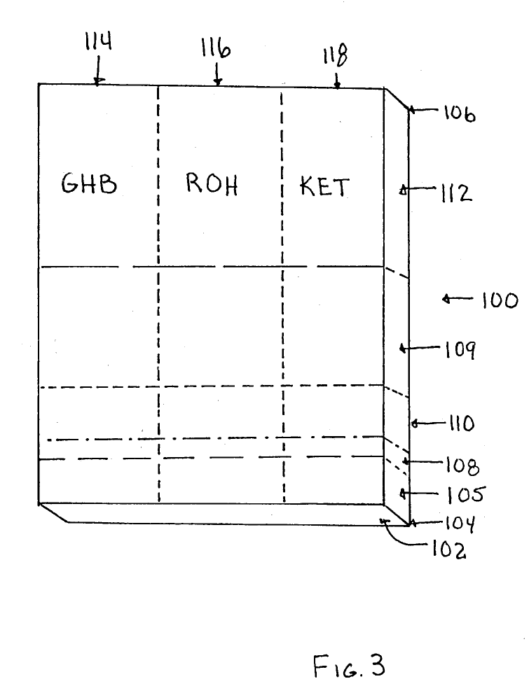

FIG. 3 shows an alternative construction for a rapid access testing system 100 according to the present invention. This is a small (e.g., from 0.5 to 8 inches along each edge) packet that can be inserted into a beverage or beverage sample. There is an opening 102 at the distal end 104, while the system 100 is gripped at the proximal end 106. The system 100 is segmented into an open area 105, gross filter area 108, active ingredient supply area 110, visual indicator area 109, and auxiliary area 106. There are three horizontal zones 114 (for GHB indication), 116 (for rohypnol identification), and 118 (for ketamine identification). Other segments or other drug identification segments may be provided.

Although, the shell of the device appears in the Figures to have been manufactured of like materials, there may be structural differences between segments (as where compressibility is needed). The shell may demand usage of more than one type of plastic. Materials such as, but not limited to, polyesters (e.g., polyethylene terephthalate), polyethers, polyurethanes, polyacrylates (including methacrylates), polyolefins (polyethylene, polypropylene), polyamides, polyethylenically unsaturated materials (e.g., polystyrene, polybutadiene), vinyl resins (polyvinyl chloride, polyvinylidene chloride, polyvinyl acetals [polyvinyl butyral]), polycarbonate, polysiloxane resins, epoxy resins, cellulosic resins (cellulose acetate, cellulose acetate butyrate), and the like. It is minimally desirable that the plastic (or glass) over the display area by transmissive of visible light, at least translucent to visible light, and be insoluble in the intended liquids to be analyzed.

The device is inserted into a container, glass or cup, or placed onto a spilled out sample of beverage. When the liquid is drawn into the tube, it is observed for the requisite period of time. Once the purposeful uptake of suspect fluid is accomplished, applied pressure against the tube may be timely applied, if needed. In other constructions, mere passage of time and observation is required.

A presumptive color assay for rohypnol is described herein, a recorded color change from white to a violet/purple color occurs (if present) within 30 seconds or less. If a violet/purple color does not appear in embrasure 24 within 30 seconds, the beverage analysis should be considered negative for rohypnol (a keto-benzodiazepine). The presumptive color assay for GHB will be also be seen in the display area. The recorded color change from white to a yellow/orange color or a purple/black color also occurs within 30 seconds or less. If a yellow/orange color appears within 30 seconds or less, the beverage analysis should be considered negative for GHB. However, if a purple/black color appears within 30 seconds or less, the beverage analysis should be considered suspect for the presence of GHB. As an alternative, a peroxidase activity assay may be prepared, with an oxidizing agent, as described in WO 97/39142. That reference is incorporated by reference to teach mechanisms for designing compositions for rapid and heightened responses in such assays. These assays may be designed for individual and particular drugs. The reaction causes a chemiluminescent response that may be monitored. The system would contain a key or label for a particular enzyme designed to be responsive to the presence of the particular drug. A chemiluminescent cyclic diacylhydrazide, and azine enhancer and at least one oxidizing agent would be present. Upon sensing the target drug, the enzyme would catalyze the chemiluminescent reaction of the other ingredients (according to WO 97/39142) and chemiluminescence would be displayed in the system. This would be somewhat slower (e.g., 2-5 minutes) as compared to other systems, but would be of clinical accuracy.

Ketamine may be identified by providing a material that couples with ketamine to form a specific color dye. A wide source of underlying materials may be provided from photographic color couplers. These materials are well known in the art to provide a wide range of colors. For example, an acetylanilide-type coupler is used to form a yellow-colored image, a 5-pyrazolone-, pyrazolotriazole-, cyanoacetophenone- or indazolone-type coupler is used to form a magenta-colored image; and a phenol-type coupler, and phenol or naphthol as well, is used to form a cyan-colored image. These dye-forming intermediates (known in the art as couplers) can be provided as encapsulated oxidized forms (so they readily react upon aqueous contact with the ketamine), or can be provided in their more stable neutral form in the presence of a strong oxidizing compound that becomes active when in contact with an aqueous solution (e.g., the beverage). In this way, the activated oxidizing material (e.g., a peroxide, borate or the like) will oxidize the coupler which will in turn react with the ketamine to form a dye of a specific color. It does not matter if the oxidizing agent subsequently bleaches the dye, as the appearance of color for a brief period of time is sufficient to provide the necessary warning.

The tube elements of the invention also offer themselves to ease of use, convenience in availability, and numerous marketing capabilities. As noted, at least the display area of the element should be transparent, but it may be colored. The entire tube element may be of uniform or varied color and may be used as an accessorizing element. For example, a bangle bracelet, pendant, brooch, necklace or other decorative element may contain one or more of the tube elements. These can be easily removed by simply pulling them or unscrewing them from a holder. Little elongate jars or containers may be provided to hold them while acting as a key chain holder. Other functions may be provided on such a key chain holder. For example, an alarm or signal may be provided with the holder so that upon sensing the presence of a drug, especially if some of the beverage has been consumed, the alarm may be sounded. The sensors may be provided as swizzle sticks (since there is little or no chance of any reflux of materials back into the beverage, or the TLC construction has chemically inert materials such as silica, titania, zirconia (or other metal oxides) or crosslinked polymeric beads as the chromatographic beads. Male screw-in ends may be provided, so that the elements may be easily removed from other devices. As the tube elements may not be susceptible to repeated use, the elements may be removed and replacement elements reinserted into the male screw-in tips.

The following examples are non-limiting examples intending to exemplify practice of the invention and not to limit the invention to less then the generic practices described above.

### EXAMPLE 1

The following GHB indicator ingredients are combined, as now described:

The crystalline indicator 5,5′ Dibromo-o-cresolsulfonphthalein is dissolved in Ethanol, 80% v/v to a level of (5%) five percent by weight. It is then encapsulated in a thin gelatin microcapsule according to known commercial processes.

Referring to FIG. 1, the microcapsules are positioned within a hollow 100 mm tube, behind a coarse filter paper mask, set 2 mm away from a liquid receiving end. The unique reaction between BCP or bromcresol purple (5,5′ dibromo-o-cresolsulfonphthalein) and GHB or sodium gamma hydroxybutyrate acid resulting in color species will occur when the tube is inserted in the beverage, liquid filtered through the coarse filter, and the microcapsule dissolves (thirty seconds) to release the bromocresol purple. When combined, BCP and GHB form a strong color complex. The term coarse filter does not intend any functional limitation on the practice of the invention, as noted earlier in the description. It is to be distinguished only from any refined separation method that would actually isolate individual chemicals for testing in a separate location (other than by the TLC method, which is a straight-line method that essentially segments the drugs and ingredients.

### EXAMPLE 2

The following rohypnol indicator ingredients are combined, as now described in the similar construction. Zimmermann's Test Reagents for Diazepam. The liquid indicators Reagent A (methanolic 2,4-dinitrobenzene) and Reagent B (potassium hydroxide solution) are prepared commercially. The specimen of beverage must come in contact with Reagent A first, preparing the specimen. Then contact with Reagent B will visually qualify the specimen for keto-benzodiazepines. Additional indicators are presently under development and show substantial promise with less concerns for their toxic attributes.

The element should be constructed to accept the varying degrees of temperature ranges associated with common beverages ingested by humans. A flexible polymer is indicated for beverages in temperate liquids, and a plastic stick form or crosslinked polymer, or even glass form is also illustrated for beverages of higher temperatures. However, as should be understood from the above description, the device does not need to be immersed for an extended period of time to be effective.

The dimensions of the structure are by way of example only. The structure should not be, in any way, restricted to the dimensions conveyed here. For the specific dimensions may be attributed to the analytical variations arising while formatting a specific strip to identify a unique substance. Conveniently the tube is at least 4 cm long, up to 30 or 40 cm, maximum length. The length above 4 cm is not functionally limiting, but is merely one of convenience. The length below 4 cm also tends to be one of convenience, as a device of 1 cm could be used. The thickness is again more of convenience rather than functionality. Elements of less than 0.1 mm would be relatively slower and less noticeable than one with a diameter of 0.3 mm and higher, with an element greater than 2 cm being inconvenient. A preferred range would therefore be in the range of 0.3 to 1 cm in thickness.

Typically, the support ranges in thickness from about 0.2 to about 0.08 inches; however, as will be understood, the thickness may, if suitable, vary from this range. The support can be made of thermoplastic materials that after curing do not give off toxic resides and that can be produced and packaged in a sterile environment. Materials for the preferred design criteria and production of this device are readily available. For example, the support medium may be constructed from a combination of two suitable thin, flat films of synthetic material conventionally employed for such purposes. The films are not porous and, after permanently being joined, will provide a liquid impermeable support and chamber within.

Suitable materials for the film are, for example, polyethylene, polyurethanes, polysiloxanes, polypropylene, polyvinyl type polymers, or copolymers, such as polyvinyl chloride, polyvinyl acetate and the like. It is preferable that the support be of the material Ultra-High Molecular Weight Polyethylene which is Food and Drug Administration approved for food contact applications

Advantageously, Ultra-High Molecular Weight or High Density Polyethylene provide good chemical and environmental resistance, are non-toxic and have natural white color but, as can be readily appreciated, the material and color may be changed. This indicator may be organic or inorganic, but in any event will be suitable for analyzing the beverage. The indicator suitably present on reagent carrier 45 is in an amount sufficient to ensure detection by achieving a strong color complex.

The indicator useful for a presumptive color assay for the detection of Sodium Gamma Hydroxybutyric Acid (C₄H₇O₃Na) in a beverage is: 5,5′ Dibromo-o-cresolsulfonphthalein. The reagent solution matrix for GHB color indication is the same as previously described.

The preferred indicator useful for a presumptive color assay for the detection of the pharmaceutical Flunitrazepam (C₁₆H₁₂FN₃O₃) in a beverage are: Zimmerman's Test Reagent (methanolic 2,4-dinitrobenzene) and (potassium hydroxide). The reagent solution matrix for Flunitrazepam color Indication is as previously described.

Variations of the practice of the invention may be seen in the following descriptions, and other variations may be practiced by one generally skilled in the art. A tube or tube-like element (any cross-section may be used, such as circular, oval, triangular, rectangular and the like) havs a distal end and a proximal end (which is usually placed into the liquid or used to withdraw a small amount of liquid), the distal end having an opening to a lumen within the tube. The lumen within the tube has a visual indicator area specific to the presence of at least two different drugs selected from the group consisting of rohypnol, gamma hydroxyl butyrate, and ketamine. Other drugs may have their own indicating areas and materials, but these are presently the most common hypnotic or anesthetic drugs that pose a threat to the public. The visual indicator area provides a visual indication of the presence of at least two of these hypnotic or anesthetic drugs in a beverage without the addition of any other reactants to the lumen other then those carried in the beverage. The visual indication may include a marking spot indicative of at least one of the drugs along a color indicating strip, in a rate movement test as with blotting paper, pH indicator paper, chromatographic paper and the like. The visual indication area may include a chemilumenescent response indicator in response to at least one of the at least two drugs or more than one of the drugs. The chemilumenescent response may comprise a peroxidase activity response. The visual response area may provide at least one reagent for effecting a chemical reaction with a drug to form a dye or pigment different in color from the reagent. This will provide for a clear color change reaction specific to one of the classes of hypnotic or anesthetic drugs. The indicator dyes may be present at specific marking positions along a strip through which liquid in the beverage may flow, showing the color change at specific positions on the strip to reduce the potential for false positives. The indicator dyes should be responsive to particular drugs that would appear at those marking positions if present during a test with imbibing of beverage into a porous strip.

A testing device for the presence of drugs in a liquid according to the invention may comprise an elongate tube with at least two different reactants therein, each of the reactants being specific to the formation of different colors with a specific hypnotic or anesthetic drug. The tube may have an open distal end and a proximal end, the distal open end leading to a lumen. The lumen may contains, in order moving away from the end that is placed in the liquid, a physical filter and a display zone, the display zone providing a visual indication of the presence of at least two hypnotic or anesthetic drugs, preferably those selected from the group consisting of rohypnol, gamma hydroxyl butyrate, and ketamine, without the addition of any other reactants to the lumen except those carried in the liquid. This avoids the need for separate testing kits, technical manipulation of multiple reagents, and assures more rapid and facile use of the testing kit.

The physical filter is preferably set off from the distal end by a distance of at least 0.5 mm to provide a spacing between the filter and the drink so that liquid first is drawn into the tube, then contacts the filter, and filtered liquid (with potential dissolved drug therein) moves along the tube to the indicating area. The physical filter is set off from the distal end by a distance of at least 1.0 mm, and the display zone may comprise a porous layer that follows the filter layer. An ingredient supply zone may be present that provides an ingredient for assistance in the visual indication is intermediate the distal end and the porous layer, such as a dye or oxidizing agent or educing agent. Such an ingredient supply zone that provides an ingredient for assistance in the visual indication may be intermediate the distal end and the display zone. A portion of the tube element or all of the tube element may be compressible to allow for elastic flexing of sides of the tube by application of external force or allow squeezing to effect some suction effect into the tube. Elastic flexing of the tube may rupture frangible containers within the lumen to release a material that assists in providing a visual indication of the presence of at least one of the drugs.

A single elongated tube with adjacent or alternating segments with drug testing capability may also be provided. For example, an elongated tube would have multiple segments of tubing. At least two of the segments would each have a visual indicator area specific to the presence of at least two different hypnotic drugs or anesthetic drugs. The segments of the tube would be separable from each other, so that a segment could be broken off and used, and then the next segment could be broken off and used. This could be easily manufactured, for example, by providing a strip of paper with the three drug testing components parallel and adjacent each other along the length of the tube. The paper could be inserted into a tube (e.g., such as a transparent stirring stick). Crimps could be placed at various intervals along the filled stirring stick. Whenever a test was desired, a different segment of the stick could be broken off and that segment contacted with the beverage/liquid.

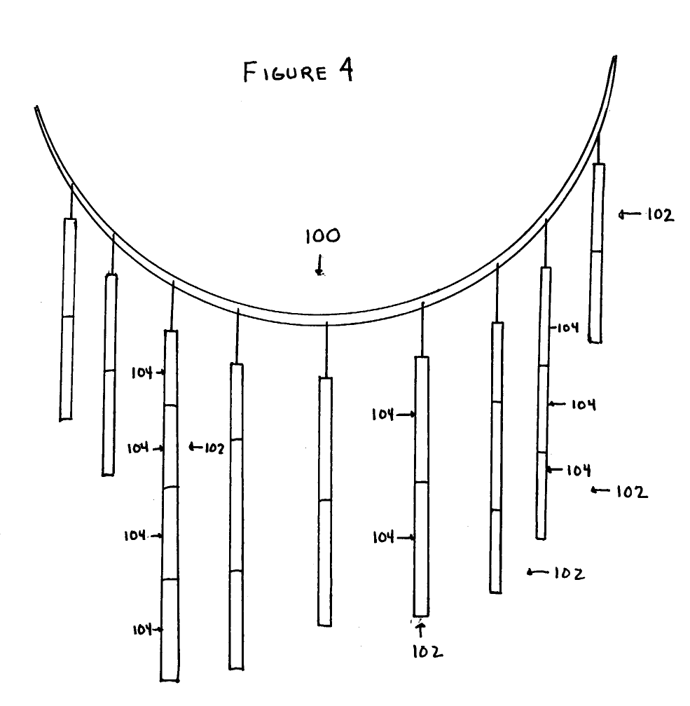

Multiple stick (either single test or multiple test (as described immediately above) could be provided as stem elements on a necklace or bracelet so they are available and relatively inconspicuous. This is shown in FIG. 4. A necklace 100 has multiple stems 102 hanging therefrom. Each stem 102 has individual segments 104 that comprise testing areas for at least two drugs. Each segment 104 can be separated from an adjacent segment for test use.

What is claimed:

1. A tube having a distal end and a proximal end, the distal end having an opening to a lumen within the tube,
the lumen within the tube having a visual indicator area specific to the presence of at least two different drug selected from the group consisting of rohypnol, gamma hydroxyl butyrate, and ketamine,
the visual indicator area providing a visual indication of the presence of at least two of these drugs in a beverage without the addition of any other reactants to the lumen other then those carried in the beverage.

2. The device of claim 1 wherein the visual indication includes a marking spot indicative of at least one of the drugs along a color indicating strip.

3. The device of claim 2 wherein the visual indication area includes a chemilumenescent response indicator in response to at least one of the at least two drugs.

4. The device of claim 3 wherein the chemilumenescent response comprises a peroxidase activity response.

5. The device of claim 1 wherein the visual response area provides at least one reagent for effecting a chemical reaction with a drug to form a dye or pigment different in color from the reagent.

6. The device of claim 2 wherein indicator dyes are present at specific marking positions along a strip through which liquid in the beverage may flow.

7. The device of claim 6 wherein the indicator dyes are responsive to particular drugs that would appear at those marking positions if present during a test with imbibing of beverage into a porous strip.

8. A testing device for the presence of drugs in a liquid comprising an elongate tube with reactants therein, the tube having an open distal end and a proximal end, the distal open end leading to a lumen,
the lumen contains, in order, a physical filter and a display zone, the display zone providing a visual indication of the presence of at least two drugs selected from the group consisting of rohypnol, gamma hydroxyl butyrate, and ketamine, without the addition of any other reactants to the lumen except those carried in the liquid.

9. The device of claim 8 wherein the physical filter is set off from the distal end by a distance of at least 0.5 mm.

10. The device of claim 8 wherein the physical filter is set off from the distal end by a distance of at least 1.0 mm.

11. The device of claim 10 wherein the display zone comprises a porous layer that follows the filter layer.

12. The device of claim 11 wherein an ingredient supply zone that provides an ingredient for assistance in the visual indication is intermediate the distal end and the porous layer.

13. The device of claim 8 wherein an ingredient supply zone that provides an ingredient for assistance in the visual indication is intermediate the distal end and the display zone.

14. The device of claim 8 wherein a portion of the tube element is compressible to allow for elastic flexing of sides of the tube by application of external force.

15. The device of claim 8 wherein elastic flexing of the tube ruptures frangible containers within the lumen to release a material that assists in providing a visual indication of the presence of at least one of the drugs.

16. A tube having a distal end and a proximal end, the distal end having an opening to a lumen within the tube,
the lumen within the tube having a visual indicator area specific to the presence of at least two different hypnotic drugs or anesthetic drugs,
the visual indicator area providing a visual indication of the presence of at least two of these drugs in a beverage without the addition of any other reactants to the lumen other then those carried in the beverage.

17. The tube of claim 16 comprising an elongated tube having multiple segments of tubing, at least two of the segments each having a visual indicator area specific to the presence of at least two different hypnotic drugs or anesthetic drugs, the segments of the tube being separable from each other.
